(19) United States
(12) Patent Application Publication
(10) Pub. No.: US 2001/0049486 A1
(43) Pub. Date: Dec. 6, 2001

(54) Method and system for re-infusing filtered bodily aspirates

(76) Inventors: Michael A. Evans, Palo Alto, CA (US); Denise M. Demarais, San Jose, CA (US); Stephen A. Leeflang, Stanford, CA (US)

Correspondence Address: Mark D. Barrish, TOWNSEND and TOWNSEND and CREW LLP, Two Embarcadero Center, 8th Floor, San Francisco, CA, 94111-3834, US

(21) Appl. No.: 09/751,216

(22) Filed: Dec. 29, 2000

Related U.S. Application Data
(60) Provisional application No. 60/174,108, filed on Dec. 31, 1999.

(51) Int. Cl. A61M 1/38

(52) U.S. Cl. 604/4.01; 604/5.01; 604/6.09; 604/6.1; 604/6.12; 604/19; 604/35; 604/500

(57) ABSTRACT

Methods and apparatus filter and reinfuse aspirate removed from body lumens following a medical procedure. Evacuation of harmful material such as blood clot or plaque from a blood vessel can be combined with filtering of blood from the unwanted material, allowing the blood to be reinfused back into the patient's system through various means, thereby minimizing the effects of blood loss to the patient.

### CROSS-REFERENCES TO RELATED APPLICATIONS

The present application is a regular patent application of and claims the benefit of priority from U.S. patent application Ser. No. 60/174,108 filed Dec. 31, 1999 (Attorney Docket No. 19744P-000900), the full disclosure of which is incorporated herein by reference.

### BACKGROUND OF THE INVENTION

The present invention relates generally to medical apparatus and methods and more particularly to devices and methods for re-infusing bodily aspirates. In one embodiment, the aspirates are re-infused following a procedure such as thrombectomy, embolectomy, atherectomy or other procedures utilizing aspiration for removal of unwanted material such as thrombus, atheroma, fluid, polyps, cysts or other obstructive matter from body lumens, such as blood vessels, ureters, bile ducts or fallopian tubes.

Sometimes during these procedures, and in particular during procedures in the blood vessel, it is desirable to decrease the blood loss resulting from aspiration, by either supplementing the patient's blood supply with an infusion of donor blood or using their own blood that has been cycled through a blood purification system such as a device known as a “cell saver”. Since the advent of more minimally invasive procedures utilizing catheters, less invasive methods for minimizing blood loss have been developed such as better catheter valves and better techniques to hasten procedure time and thereby lessen blood loss.

In the case of some procedures, such as aggressive thrombectomy where a large volume of blood clot and blood can be removed through the combination of maceration coupled with infusion and aspiration of material, known devices are not sufficient to achieve the desired blood loss containment. For example, using devices such as those described in co-pending patent application Ser. No. 09/454,517, it would be desirable to minimize excessive blood loss while still allowing for rapid aspiration and removal of increased volumes of material to reduce procedure time. It would furthermore be desirable to supplement procedures such as this with a way to reinfuse blood that has been aspirated from the treated blood vessel, but has been filtered to remove any harmful clot material, while giving the patient the benefit of his or her own healthy blood product. The devices, systems, and methods of the present invention will find applications with a variety of thrombectomy catheters, atherectomy catheters, and other structures for removing solid materials from blood vessels and other body lumens. Exemplary thrombectomy catheters are also described in co-pending U.S. patent application Ser. Nos. 09/590,915 and 09/491,401; the full disclosures of which are incorporated herein by reference.

There is a need for an improved method and device for more efficient evacuation of harmful material such as blood clot, and for allowing the healthy blood product to be filtered and reinfused into the patient's system to decrease the effects of increased blood loss.

Preferably, such improved devices will be adapted to be used with commercial catheter devices and standard sheaths, and will be connected to those devices through standard luer fittings and tubing. Furthermore, such devices will provide an external filter system with a filter mechanism (e.g. outside the body of the patient) sufficient to filter the blood product greater than 20 microns. Additionally, such devices shall allow for the sterile re-infusion of blood product into a patient, and shall in some cases provide for isovolumetric action whereby the volume of blood and material that is aspirated is equal to that blood and fluid that is filtered and re-infused.

These objectives will be met by the design and use of the present invention.

### DESCRIPTION OF THE BACKGROUND ART

U.S. Pat. Nos. 6,001,118, 5,827,324 and 5,814,064 disclose various catheter emboli capturing and filtering devices to protect the distal portion of the blood vessel into which they are inserted. U.S. Pat. No. 5,911,734 describes a filter device having an expansion frame that is deployed as part of a guidewire system to filter and/or block unwanted material from traveling downstream in the treated artery. U.S. Pat. No. 5,895,399 describes an arterial catheter system for treating arterial stenosis, including a filter system to trap unwanted particles.

In the area of blood filtration systems, U.S. Pat. No. 5,989,438 describes a filter that actively removes particles from blood, the system including a housing, a blood inlet area, and certain reservoirs and valves to direct the blood flow and filter out the unwanted particles. Further, U.S. Pat. No. 5,540,841 describes blood filtering during cardioplegia, and U.S. Pat. No. 5,979,669 describes a glass fiber blood filtering system to filter blood through a microporous mesh. PCT Publication No. WO 00/56391 and U.S. Pat. No. 6,117,124 may also be relevant. All of these references, and all other references cited herein, are incorporated herein by reference.

In addition, certain blood filtration systems are available commercially, including the Cell Saver® and CollectFirst® systems from HAEMONETICS CORP. to salvage the patient's own blood before, during and after surgery, so that it can be reinfused thereby eliminating the need for donor blood. Pall Medical also provides liquid filtration systems such as the Supor® membrane to assist in microfiltration.

### SUMMARY OF THE INVENTION

According to the present invention, improved methods and apparatus are provided for capturing bodily fluid lost during certain medical procedures, and filtering of unwanted particles from said fluid thereby allowing said filtered fluid to be reinfused into the patient during the same procedure.

In particular, the present invention provides a solution to the problem of blood loss associated with performing mechanical thrombectomy and other such procedures in blood vessels where increased blood loss may occur due to infusion and aspiration of blood clot, plaque and other unwanted material. During a procedure such as mechanical thrombectomy, blood and macerated clot can be aspirated using a mechanical thrombectomy device and is collected in a syringe or collection bag. This effluent can then be filtered by using an inline filter to remove any dangerous debris and re-infused into the patient during the same procedure. The re-infusion can be either directly back into the treatment site, or into a secondary location such as an adjacent vein. In the vein, any small clot particles would flow into the pulmonary bed which can tolerate larger particle sizes and therefore reduce the risk of adverse events to the patient.

The preferred embodiment of the filter catheter system of the present invention includes a syringe or reservoir or other suction device to remove and contain aspirate, said reservoir being connected to an aspiration port on the treatment catheter by way of tubing and a 3-way valve. Said system further comprises an inline filter (varying porosites), an infusion device such as a syringe, and a second 3-way valve connected by catheter tubing to an infusion port on the treatment catheter or hemostasis sheath.

In operation, the filter system allows for 1) the controlled aspiration of body fluid such as blood and clot material from a blood vessel, 2) the filtration of such material to remove any unwanted blood clot particles, and 3) the reinfusion of the filtered material back into the patient to minimize the amount of blood product lost during the procedure.

The present invention may also incorporate a second catheter connected to the aspiration and filter mechanism for infusion of the filtered material to a secondary location in the body. Furthermore, the system of the present invention may be “closed loop” design, wherein a mechanism controls the following relationship: the aspirated material removed from the lumen is in volumetric proportion to the filtered material that is reinfused.

In one particular aspect, the invention provides an aspiration/reinfusion system for use with a thrombectomy catheter for removing clot from a blood vessel of a patient. The thrombectomy catheter will typically have a proximal end, a distal end, and a clot removal lumen. The system comprises an aspirator in a first fluid path from the proximal end of the clot removal lumen so that the aspirator and thrombectomy catheter draw the clot and fluid from the blood vessel. A filter is disposed in a second fluid path from the aspirator. The second fluid path extends from the aspirator to the patient. An infusor is disposed along the second fluid path for reinfusing the filtered fluid from the filter into the patient.

The aspirator will optionally comprise a vacuum source coupled to a reservoir. An exemplary aspirator may comprise a displacement pump such as a syringe, with the vacuum source comprising a piston of the syringe, and the reservoir comprising the cylinder of the syringe. Alternative vacuum sources may also be used to draw the fluid and clot from the thrombectomy catheter.

In many embodiments, at least one valve will be disposed along the first fluid path and/or the second fluid path. The valve or valves may inhibit flow from the second fluid path toward the aspirator during aspiration of the clot, and/or may inhibit flow from the infusor toward the aspirator during reintroduction of the filtered fluid. In the exemplary embodiment, two 3-way valves are used. The first valve selectively provides fluid communication between a reservoir of the aspirator and the first fluid path, or between the reservoir of the aspirator and the second fluid path. The second 3-way valve is arranged to provide selective fluid communication between either a reservoir of the infusor and the reservoir of the aspirator, or between the reservoir of the infusor and the patient along the second fluid path.

The infusor may comprise a displacement pump, typically comprising a syringe. The filter may be disposed along the second fluid flow path between the aspirator and the infusor. When the thrombectomy catheter extends into an artery of the patient for removing clot material therefrom, an infusion catheter may provide fluid communication between the infusor and a vein of the patient. The blood vessel may comprise an artificial blood vessel, such as dialysis graft. The first and second fluid flow paths may be defined by tubing, with a first length of tubing often defining the first fluid path, a second length of tubing defining a portion of the second path between the aspirator and the infusor, and a third length of tubing defining a second portion of the second fluid path between the infusor and an infusion catheter, the thrombectomy catheter, or some other fluid introduction device.

In another aspect, the invention provides an aspiration/reinfusion system for use with a catheter for removing solid material including clot or plaque from a block body lumen of a patient. The catheter has a proximal end, a distal end, and removal lumen. The system comprises aspiration means in fluid communication with the proximal end of the removal lumen for drawing the solid material and fluid from the catheter. A filter is disposed along a fluid path from the aspirator. Infusion means for reintroduction for reintroducing the filtered fluid from the filter into the patient are disposed along the fluid flow path.

In another aspect, the invention provides a method for reinfusing fluid removed from a blood vessel of a patient. The clot is removed from the blood vessel during a thrombectomy by a catheter. The method comprises drawing the clot and fluid from the catheter. The fluid is filtered from the clot outside of the patient. The filtered fluid is introduced back into the patient.

### BRIEF DESCRIPTION OF THE DRAWINGS

FIG. 1 illustrates the system components including the aspiration port on the treatment catheter, a syringe or other suction device and reservoir, a 3-way valve positioned between the aspiration port and the suction device, an in-line filter connected to a second 3-way valve by catheter tubing, which is connected to an infusion device that is in turn connected to an infusion port on the treatment catheter.

FIG. 1A illustrates a detailed construction of the in-line filter mechanism.

FIG. 2 illustrates the injection of contrast material into the lumen of a vessel.

FIG. 3 illustrates the initial stage of aspiration using the suction device.

FIG. 4 illustrates a completed aspiration wherein the reservoir of the suction device is filled with the aspirated material.

FIG. 5 illustrates injection of the aspirate through the in-line filter toward the infusion device, leaving any unwanted particulate within the filter section.

FIG. 6 illustrates the reinfusion of the filtered aspirate.

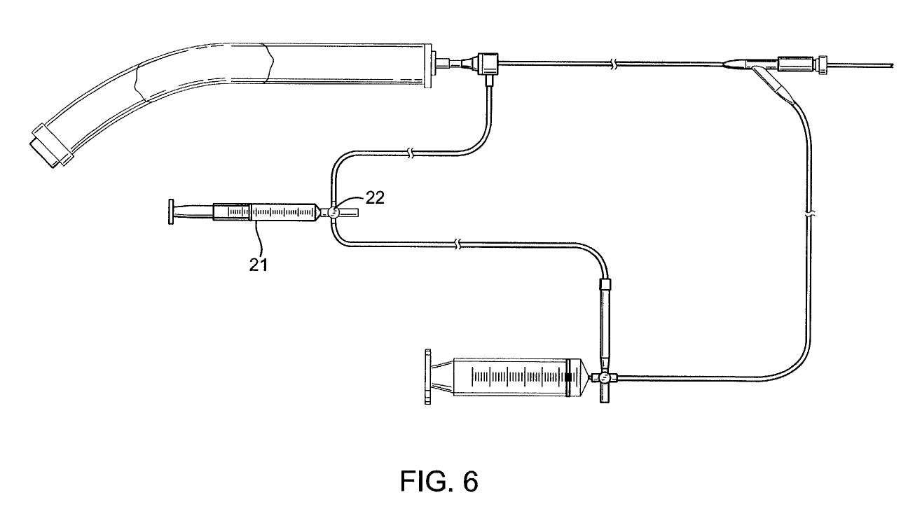

FIG. 7 illustrates an alternative embodiment, wherein the aspirate is reinfused through a second catheter system that is inserted into the vein.

### DESCRIPTION OF THE SPECIFIC EMBODIMENTS

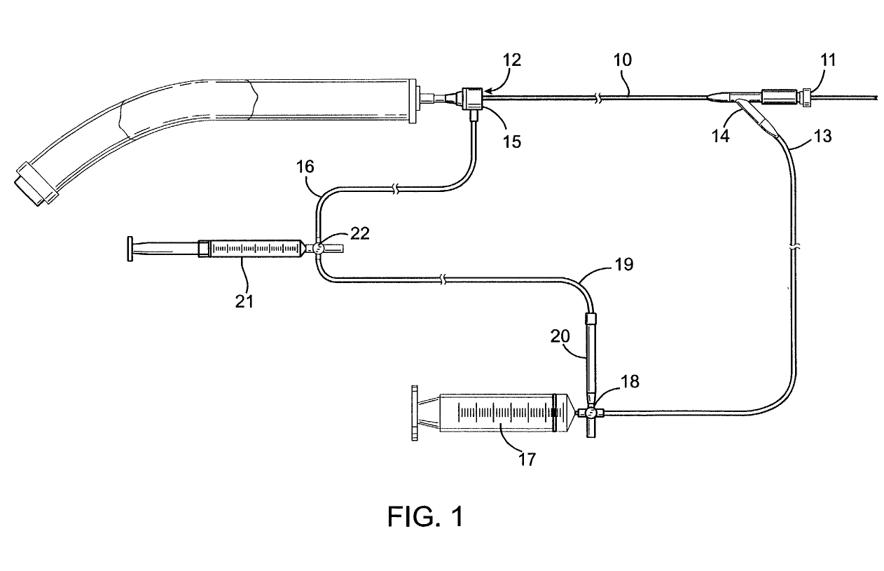

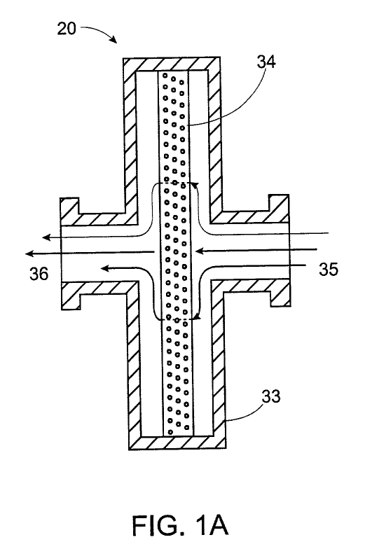

An exemplary filtration system constructed in accordance with the present invention is illustrated in FIG. 1. The system is adapted to be connected to a catheter treatment device 10 both at the aspiration port 11 and the infusion port 12 by connecting the aspiration tubing 13 and infusion tubing 16 to their respective ports on the treatment catheter through standard luer fittings such as a Y-adapter 14, available from MEDEX, or a sheath port 15, such as that commonly found on catheter sheath devices such as the Percutaneous Sheath Introducer Set available from ARRow, INC. The aspiration tubing 13 is further connected to a suction device 17 and reservoir 17¹ by a 3-way valve connector 18 such as that manufactured by Medex, and to filter catheter tubing 19 by way of said same 3-way connector. Said suction device and reservoir may be a syringe or other device such as a source of mechanical suction that can be operated to create a vacuum sufficient to pull material from the vessel to be treated. Filter catheter tubing 19 is provided with an in-line filter device 20, further described in FIG. 1A below. Filter catheter tubing 19 is then connected to an infusion reservoir 21 by an additional 3-way valve 22. Infusion reservoir 21 is linked to infusion catheter tubing 16 through the third port of 3-way valve 22. The tubing used in the filter system can be made of PVC, or Silicone, and is preferably 0.050″ to 0.200″ in diameter depending on the amount of material to be aspirated and the rate at which the desired treatment is to take place.

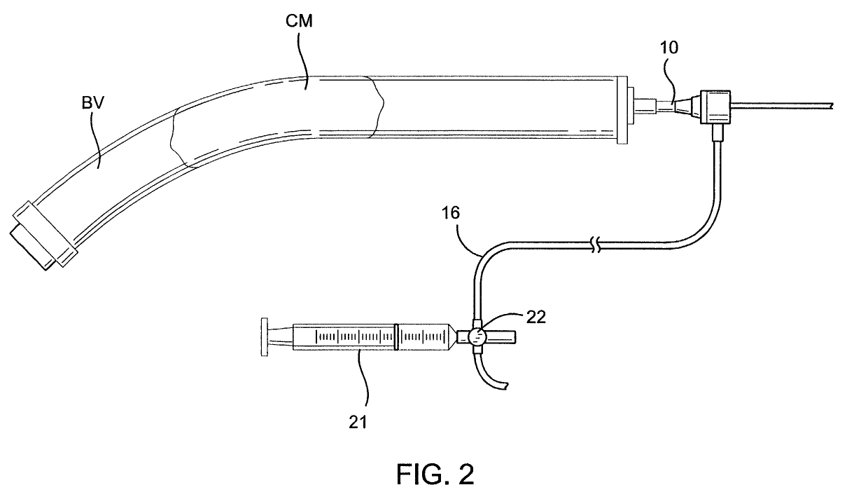

In operation, as depicted in FIG. 2, the treatment catheter 10 is percutaneously inserted into a blood vessel (BV) through an optional introducer sheath (not shown), and contrast agent is injected into the lumen through the infusion catheter tubing 16 so that the clot material (CM) to be removed from the lumen can be visualized and the treatment catheter inserted to the appropriate position. In the example shown, the infusion reservoir holds contrast, but following the injection of contrast and before the step of aspiration and infusion, a new reservoir (either empty or containing a saline solution) can be attached to the 3-way valve in preparation of receiving filtered aspirate for reinfusion.

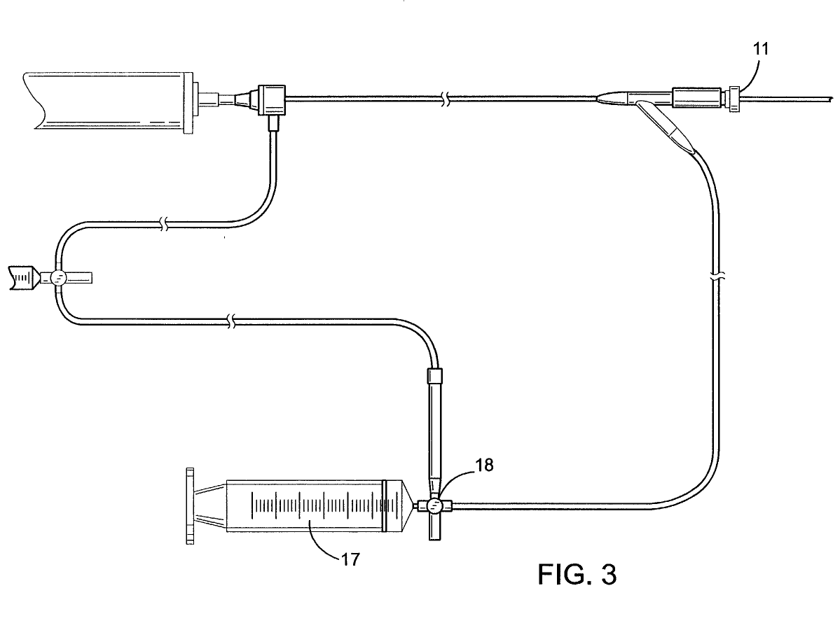

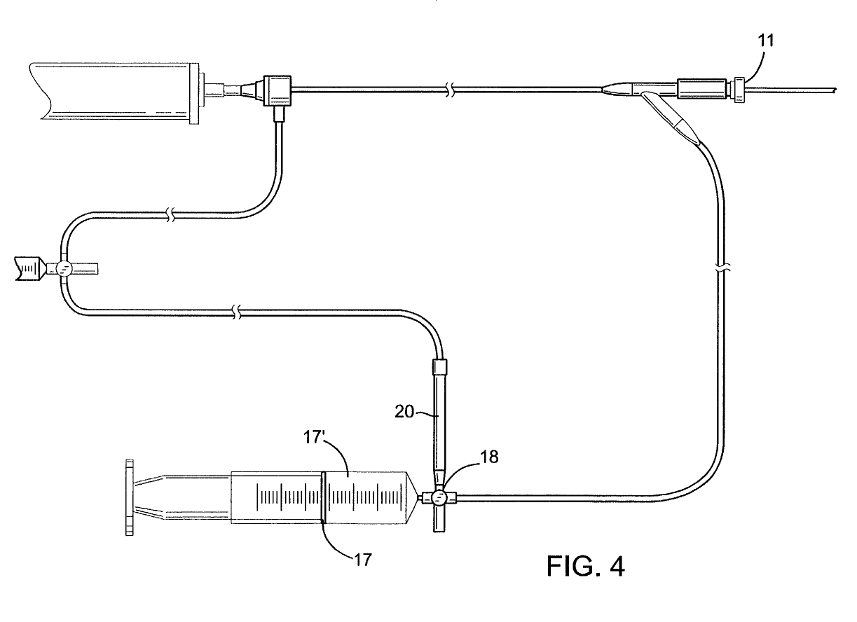

As illustrated in FIG. 3, aspiration of the material (CM) is started by opening 3-way valve 18 so that the suction device 17 and the aspiration tubing 13 are in fluid communication. Suction device 17 is then activated and aspirate is pulled from the aspiration port 11 of the treatment catheter until the suction device reservoir is filled with blood and debris. 3-way valve 18 is then positioned such that the aspiration reservoir and the filter tubing 19 are in fluid communication as shown in FIG. 4.

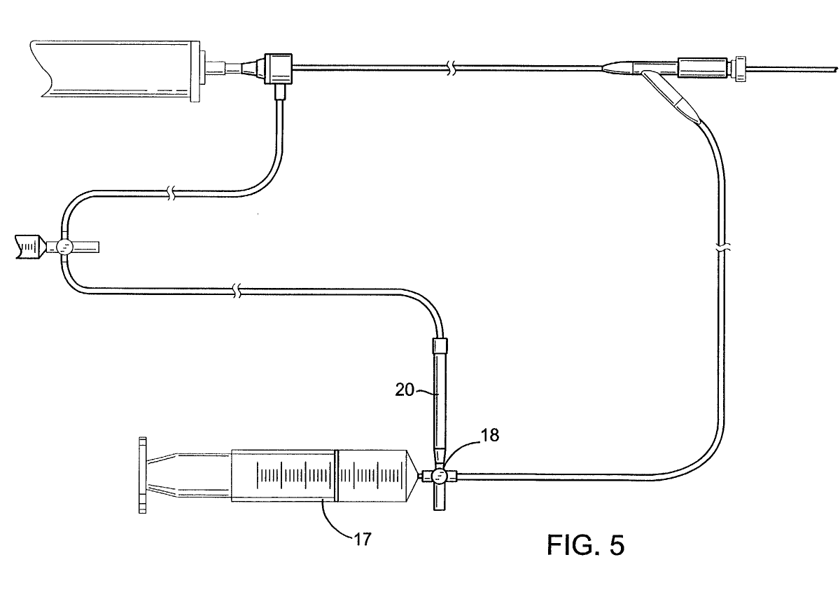

FIG. 5 illustrates the next step in the procedure wherein the aspirate from the suction device reservoir is infused into the filter catheter tubing 19 and through in-line filter 20.

Detail of the construction of the in-line filter 20 is further shown in detail in FIG. 1A. Filter 20 comprises a filter housing 33 having a filter mechanism 34 disposed therein. The filter housing provides an inflow port 35 and an outflow port 36 and a cavity for placement of said filter mechanism therebetween. Said filter mechanism may be constructed of a polymer mesh such as polyester having a range of filtration between 20 microns and 3 mm or other suitable materials such as polymer membranes, non-woven membranes, metal mesh and those typically used in blood filters.

Once the clot has been filtered out of the aspirated material, the remaining blood product can be reinfused back into the patient in several ways. Depending on the desired clinical result, the site at which the filtered blood product is reinfused can vary. In one embodiment, the blood product can be infused directly back through the infusion tubing 16 and into the infusion port 12 of the treatment catheter 10 and back into the treated area. For this embodiment, the 3-way valve 22 should be positioned such that the filter tubing and the infusion tubing are in fluid communication.

In some cases, however, this type of direct infusion is not desirable, since the treatment site may still be undergoing treatment, or may need to be left in a static condition. For example, in the case where material is aspirated from an artery, it can be dangerous to infuse back into the arterial system because of the risk that the filtered material may still contain some particulate, however small, and cause emboli or stroke in the patient, or the risk of air in the filter system being infused into the patient's arterial system. In these cases, it may be desirable to either isolate the treatment site itself to minimize complications, or reinfuse the blood product back into the patient through the patient's venous system. In the case of an isolated treatment site, the treatment catheter may have an optional expandable member such as a balloon at the distal portion of the catheter (not shown) thereby blocking blood flow distal of the treatment site. Once the unwanted material is removed through aspiration as described hereinabove, the filtered blood product can be reinfused into the isolated area without risk of emboli.

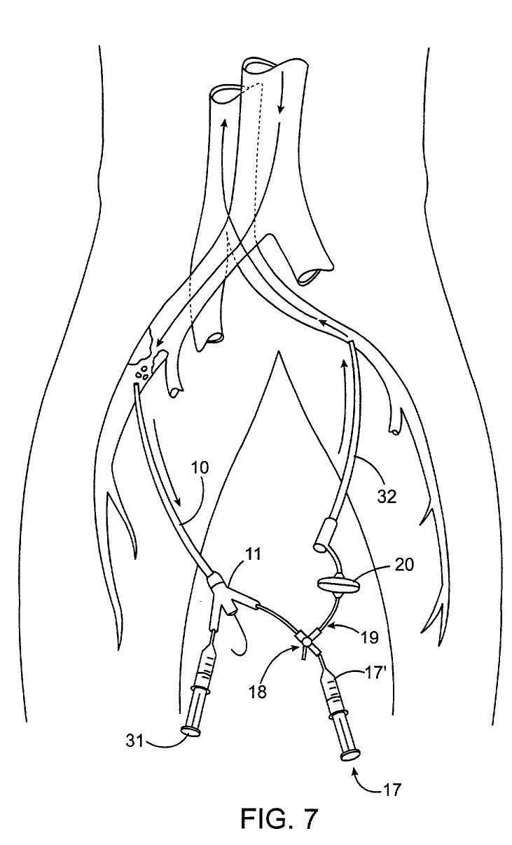

Alternatively, the venous system provides an excellent reinfusion system because the blood in the vein is pumped through the pulmonary bed which is able to filter larger particles of clot without adverse consequences. In this embodiment, as shown in FIG. 7, the infusion reservoir 31 is directly connected to the infusion port of the treatment catheter 10. The suction device 17 and reservoir 171 are connected to the aspiration port 11 on the treatment catheter, and are linked to the filter tubing 19 by a 3-way valve 18 containing an inline filter 20. In this embodiment however, instead of being linked to the infusion catheter tubing set, the filter tubing is connected to a secondary treatment catheter or hemostasis sheath 32. Said secondary treatment catheter can then be inserted elsewhere in the body away from the primary treatment site, and the blood product reinfused into the patient through that secondary site. FIG. 7 illustrates this technique in the legs of a patient, wherein the primary treatment is occurring in the artery of the leg, and the reinfusion or secondary treatment is occurring in the vein of the opposite leg.

It will often be desirable to measure, treat, and/or analyze the aspirate before reinfusion back into the patient body. For example, syringes of suction device 17 and infusion reservoir 21 can include volume markings which can be used to measure a total quantity of aspirate and/or a quantity of reinfused blood. The difference between these quantities can be analyzed to determine a particulate quantity separated by filter 20. Infusion reservoir syringe 21 may be pre-loaded with a material to treat the aspirated body fluid while the fluid is outside the body. For example, an anticoagulant such as Heparin or a contrast agent may be loaded into infusion reservoir syringe 21 to treat the blood drawn therein.

A wide variety of alternative analytical, measurement, and treatment devices and methods might be utilized. For example, an optically transparent section of tubing (or other portion of the fluid path) might be used to optionally sense particulates, to optically analyze blood characteristics, to optically measure a quantity of blood flowing along the fluid path, or the like. Weight sensors coupled to the syringes and filter 20 may also be used for measurement and/or analysis, and a third syringe coupled to the fluid path (not shown) may controllably treat the fluid. Temperature varying means (such as a resistance heater or chilled saline bath) may thermally treat the fluid. Any of a wide variety of known blood treatment and/or analysis systems might also be used.

In some embodiments, the fluid may be augmented prior to reintroducing the fluid back into the patient. Where significant blood loss has occurred, replacement blood may be infused via the infusion catheter. Saline, plasma, and/or drugs may also be introduced by augmenting the aspirated fluids prior to reinfusion. Once again, the syringes 17, 21 may be preloaded with the desired augmenting fluid, or some other fluid injector (such as a syringe) may add the augmenting fluid to the tubing.

While the above is a complete description of the preferred embodiments of the invention, various alternatives, modifications, and equivalents may be used. Therefore, the above description should not be taken as limiting the scope of the present invention.

What is claimed is:

1. A filtration system for use outside the body of a patient to filter bodily fluids and reinfuse said bodily fluids back into the patient, said system comprising:
a device capable of creating a pressure differential for aspirating bodily fluids from within a body lumen and further capable of expelling said aspirate from said reservoir, said suction device being operatively connected to an aspiration tube by way of a 3-way valve device;
a filter tube operatively connected to said suction device and said 3-way valve device, said filter tube having a filter element disposed therewithin;
an infusion tube operatively connected to said filter tube such that when said bodily fluids are expelled from said reservoir, said bodily fluids flow through said filter tube and said filter element and through said infusion tube.

2. A device of claim 1 wherein said infusion tube is operatively connected to an infusion reservoir by way of a 3-way valve.

3. A device of claim 1, further comprising means for at least one of:
measuring, treating, and analyzing the bodily fluids prior to reinfusion of the bodily fluids.

4. A device of claim 1, further comprising means for augmenting the bodily fluids with at least one of:
a drug, plasma, saline, and blood prior to reinfusion of the bodily fluids.

5. A method of filtering unwanted material such as clot, plaque and the like, from the blood product of a patient and reinfusing said filtered blood product back into the body of the patient, said method comprising the steps of:
providing a treatment catheter having an aspiration port and an infusion port and a lumen extending therebetween;
aspirating blood product and any unwanted material from the vessel of a patient through the aspiration port of said treatment catheter and into a reservoir;
infusing said blood product from said reservoir through a filter device to remove any unwanted material from said blood product and returning said filtered blood product back into the body of the patient through the infusion port of said treatment catheter.

6. A method of filtering unwanted material such as clot, plaque and the like, from the blood product of a patient and reinfusing said filtered blood product back into the body of the patient, said method comprising the steps of:
providing a first treatment catheter having an aspiration port on the sidewall thereof;
providing a second treatment catheter having an infusion port on the sidewall thereof;
aspirating blood product and any unwanted material from the vessel of a patient through the aspiration port of said first treatment catheter and into a reservoir;
infusing said blood product from said reservoir through a filter device to remove any unwanted material from said blood product and
returning said filtered blood product back into the body of the patient through the infusion port of said second treatment catheter.

7. The method of claim 6, further comprising augmenting the blood product with at least one of:
a drug, plasma, saline, and blood prior to returning the filtered blood products back into the body.

8. The method of claim 6, further comprising at least one of:
measuring, treating, and analyzing the blood products prior to returning the filtered blood products back into the body.

9. An aspiration/reinfusion system for use with a thrombectomy catheter for removing clot from a blood vessel of a patient, the thrombectomy catheter having a proximal end, a distal end, and a clot removal lumen, the system comprising:
an aspirator in a first fluid path from the proximal end of the clot removal lumen so that the aspirator and thrombectomy catheter draw the clot and fluid from the blood vessel;
a filter in a second fluid path from the aspirator, the second fluid path extending from the aspirator to the patient;
an infusor disposed along the second fluid path for reintroducing the filtered fluid from the filter into the patient.

10. The system of claim 9, further comprising means for at least one of: measuring, treating, and analyzing the fluid prior to reinfusion of the fluid.

11. The system of claim 9, further comprising means for augmenting the fluid with at least one of: a drug, plasma, saline, and blood prior to reinfusion of the fluid.

12. The system of claim 9, wherein the aspirator comprises a vacuum source coupled to a reservoir.

13. The system of claim 12, wherein the aspirator comprises a syringe, the vacuum source comprising a piston of the syringe and the reservoir comprising the cylinder of the syringe.

14. The system of claim 9, further comprising at least one valve disposed along at least one of the first fluid path and the second fluid path.

15. The system of claim 14, wherein the at least one valve can inhibit flow from the second fluid path toward the aspirator during aspiration of the clot and fluid along the first fluid path.

16. The system of 14, wherein the at least one valve can inhibit flow from the infusor toward the aspirator during reintroduction of the filtered fluid.

17. The system of claim 15, further comprising:
a first 3-way valve arranged to provide selective fluid communication between:
a reservoir of the aspirator and the first fluid path, or
the reservoir of the aspirator and the second fluid path; and
a second 3-way valve arranged to provide selective fluid communication between:
a reservoir of the infusor and the reservoir of the aspirator, or
the reservoir of the infusor and the patient along the second fluid path.

18. The system of claim 9, wherein the infusor comprises a displacement pump.

19. The system of claim 18, wherein the filter is disposed along the second fluid flow path between the aspirator and the infusor.

20. The system of claim 9, further comprising an infusion catheter for providing fluid communication between the infusor and a vein of the patient when the thrombectomy catheter extends into an artery of the patient.

21. An aspiration/reinfusion system for use with a catheter for removing solid material including clot or plaque from a body lumen of a patient, the catheter having a proximal end, a distal end, and a removal lumen, the system comprising:
aspiration means in fluid communication with the proximal end of the removal lumen for drawing the solid material and fluid from the catheter;
a filter disposed along a fluid path from the aspirator;
infusion means for reintroducing the filtered fluid from the filter into the patient along the fluid flow path.

22. The system of claim 21, further comprising means for at least one of: measuring, treating, and analyzing the fluid prior to reinfusion of the fluid.

23. The system of claim 21, further comprising means for augmenting the fluid with at least one of: a drug, plasma, saline, and blood prior to reinfusion of the fluid.

24. A method for reinfusing fluid removed from a blood vessel of a patient, wherein the fluid is removed with clot during a thrombectomy by a catheter, the method comprising:
drawing the clot and fluid from the catheter;
filtering the fluid from the clot outside the patient; and
introducing the filtered fluid back into the patient.

25. The system of claim 24, further comprising augmenting the fluid with at least one of: a drug, plasma, saline, and blood prior to reintroducing the filtered fluid.

26. The system of claim 24, further comprising at least one of: measuring, treating, and analyzing the fluid prior to reintroducing the filtered fluid.
